(19) United States
(12) Patent Application Publication
(10) Pub. No.: US 2007/0098778 A1
(43) Pub. Date: May 3, 2007

(54) Co-formulations or kits of bioactive agents

(75) Inventor: Suresh Borsadia, Plainsboro, NJ (US)

Correspondence Address: ROGER GLENN HENDRICKS, 3203 YOST, PEARLAND, TX, 77581, US

(73) Assignee: ABEILLE PHARMACEUTICALS, INC., Princeton, NJ (US)

(21) Appl. No.: 10/595,884

(22) PCT Filed: Feb. 28, 2005
(86) PCT No.: PCT/US05/06043

§ 371 Date: May 18, 2006

Related U.S. Application Data
(60) Provisional application No. 60/549,420, filed on Mar. 2, 2004.

(51) Int. Cl. A61K 31/426; A61K 31/401; A61K 31/366; A61K 31/22; A61K 31/155; A61K 9/48

(52) U.S. Cl. 424/451; 514/369; 514/423; 514/460; 514/548; 514/635

(57) ABSTRACT

Provided, among other things, is a formulation or kit comprising: (a) a pharmaceutically effective dosage of one or more a glucose-level-controlling bioactive agents selected from an α-glucodase inhibitor, sulfonylurea, meglitinide, thiazolidinediones, biguanide, insulin, dual PPARα/γ agonist, PPARγ agonist or insulin secretagogue; and (b) a pharmaceutically effective dosage of (i) one or more of an antihypertensive bioactive agent selected from an ACE inhibitor, calcium channel blocker, beta blocker, angiotension II receptor antagonist or diuretic, or (ii) one or more of an anti-dyslipidemia bioactive agent selected from a HMG-CoA reductase inhibitor, bile acid sequestrant, fibric acid derivative, sterol, cholesterol absorption inhibitor, MTP inhibitor or nicotinic acid derivative; wherein: in the case of (i) a combination of a first bioactive agent of group (a) that is metformin with a second bioactive agent of group (b), or (ii) a combination of a first bioactive agent of group (a) that is a thiazolidinedione or dual PPARα/γ agonist with an angiotension II receptor antagonist, one or more of the following applies: (I) one of the first bioactive agent or the second bioactive agent is formulated for sustained release, and the other is formulated for immediate release, each formulated for once-a-day dosing; or (II) the co-formulation or kit comprises (A) a biguanide and a thiazolidinedione and (B) one or more group (b) bioactive agents.

The present invention relates to the use of multi-bioactive agent administration products having two or more different bioactive agents indicated for two or more different disease conditions to an individual in need of such bioactive agents. More specifically, the invention relates to co-formulations or kits of two or more different bioactive agents for treating diabetes and its co-morbidities (including co-existing disease conditions), including hypertension, dyslipidemia, cardiovascular disease, and nephropathy

According to studies cited in Merck Manual, only about half of patients who leave a physician's office with a prescription take the medicament as directed. The most common reason given for noncompliance is forgetfulness. Other reasons for noncompliance with medicament regimens include lack of understanding or confusion about dosing. Older persons with cognitive impairment often find the dosing regimen complex, and difficult to remember and to follow.

Current therapeutic regimens for individuals having two or more separate disease conditions typically involve treatment with two or more different and distinct bioactive agents, which often need to be given at separate times. The individuals need to take each bioactive agent at its required time for maximum therapeutic effect. Individuals who must take multiple bioactive agents on multiple schedules to treat more than one disease condition often find this multi-bioactive agent administration regimen very confusing and even more difficult to follow. Multi-bioactive agent administration is especially difficult for elderly patients who often have several co-existing conditions needing therapeutic treatment.

Various solutions have been suggested for improving patient compliance with medication dosing, including intervention by the physician and/or pharmacist. However, it is generally accepted that the best possible dosing regimen for patient compliance is a simplified regimen, especially a once-daily dosing regimen.

There is recent evidence based on prospective and retrospective analysis that comorbidity of hyper-lipidemia and diabetes is prevalent and could benefit from concomitant treatment with an anti-dyslipidemia agent and a glucose controlling agent.

Co-administration of these bioactive agents however is often difficult since the glucose lowering agent often needs to be dosed multiple times a day and the anti-dyslipidemia agent is typically administered once a day, preferably at night time. A single administration of anti-diabetic bioactive agent can be achieved by formulating the bioactive agent in a sustained release dosage form. However, addition of the anti-dyslipidemia agent in the same sustained release dosage form could inappropriately control the second bioactive agent's release such that it would be difficult to achieve therapeutic levels.

U.S. Pat. No. 6,660,300 to Timmins et al describes a delivery system wherein Metformin and optionally a hypolipidemic agent are administered in a biphasic system. The delivery system's two phases are: an inner solid particulate phase of granules containing a highly water soluble pharmaceutical with hydrophilic and hydrophobic polymers, and an outer solid continuous phase in which the inner solid particulate phase granules are dispersed. The outer solid continuous phase is formed of an extended release material with hydrophobic polymers or materials. Since both phases in the described system contain hydrophobic polymers, it is likely to control the release of not only highly soluble Metformin which requires an extended release profile, but will also inhibit the release of any hypolipidemic agent, leading to sub-therapeutic levels of the hypolipidemic agent.

WO 2004/017896 A2, to Waldstreicher et al, describes a combination bioactive agent therapy for the treatment of hypertension and type 2 diabetes mellitus, Metabolic Syndrome, or a pre-diabetic condition in a patient in need of such treatment. The invention describes the use of combinations of pharmaceutically active compounds that are dual agonists of the alpha and gamma subtypes of the peroxisome proliferators activated receptor (PPARα/γ) with Angiotensin II Type I receptor (A-2) antagonists. The invention does not specifically address the combinations of other bioactive agents for treatment of diabetes and comorbidities, particularly where the bioactive agents have distinct treatment regimens.

What is needed then is a multi-bioactive agent administration product for concurrently treating two distinct disease conditions each of which has distinct treatment options and different treatment regimens.

SUMMARY OF THE INVENTION

Provided, among other things, is a formulation or kit comprising:
(a) a pharmaceutically effective dosage of one or more a glucose-level-controlling bioactive agents selected from an a-glucodase inhibitor, sulfonylurea, meglitinide, thiazolidinediones, biguanide, insulin, dual PPARα/γ agonist, PPARγ agonist or insulin secretagogue; and
(b) a pharmaceutically effective dosage of (i) one or more of an antihypertensive bioactive agent selected from an ACE inhibitor, calcium channel blocker, beta blocker, angiotension II receptor antagonist or diuretic, or (ii) one or more of an anti-dyslipidemia bioactive agent selected from a HMG-CoA reductase inhibitor, bile acid sequestrant, fibric acid derivative, sterol, cholesterol absorption inhibitor, MTP inhibitor or nicotinic acid derivative;
   wherein:
in the case of (i) a combination of a first bioactive agent of group (a) that is metformin with a second bioactive agent of group (b), or (ii) a combination of a first bioactive agent of group (a) that is a thiazolidinedione or dual PPARα/γ agonist with an angiotension II receptor antagonist, one or more of the following applies:
   (I) one of the first bioactive agent or the second bioactive agent is formulated for sustained release, and the other is formulated for immediate release, each formulated for once-a-day dosing; or
   (II) the co-formulation or kit comprises (A) a biguanide and a thiazolidinedione and (B) one or more group (b) bioactive agents.

The multi-bioactive agent administration product may be a co-formulation or a kit. The kit may comprise, for example, daily dosing for 7, 14, 21, 28 or more days.

In certain embodiments, such a co-formulation is a capsule wherein
one or more group (a) bioactive agents are formulated in sustained release beads comprised within the capsule; and
one or more group (b) bioactive agents in a more immediate release form are comprised within the capsule.

In certain embodiments, such a co-formulation is a compression formulation wherein
one or more group (a) bioactive agents are formulated in sustained release form comprised within a portion of the compression formulation; and
one or more group (b) bioactive agents in a more immediate release form are comprised within another portion of the compression formulation.

In certain embodiments, such a co-formulation is a suspension formulation wherein
one or more group (a) bioactive agents are formulated in sustained release form comprised within particles that are suspended or adapted to be suspended in a liquid; and
one or more group (b) bioactive agents are dissolved in the liquid.
   The instructions for the co-formulation may provide that it should be shaken immediately prior to use (to suspend particles). Particles may be beads, such as described below. Bead and liquid density can be selected to increase bead propensity to remain in suspension.

In certain embodiments the invention provides, among other things, methods of treating diabetes or its co-morbidities. One such method is for delivering in the co-formulation a glucose-level-controlling bioactive agent and a second bioactive agent for treating a co-morbidity of diabetes, the glucose-level-controlling bioactive agent having a first dosing regimen and the second bioactive agent having a second, distinct dosing regimen, wherein the co-formulation provides a pharmacokinetic profile of the glucose-level-controlling bioactive agent that mimics the first dosing regimen and a pharmacokinetic profile of the second bioactive agent that mimics the second dosing regimen.

### BRIEF DESCRIPTION OF THE DRAWINGS

FIG. 1 is an embodiment of a kit of the invention.

FIG. 2 is another embodiment of a kit of the invention.

FIG. 3 is a further embodiment of a kit of the invention.

FIG. 4 shows dissolution profiles of formulations of the invention.

### DETAILED DESCRIPTION OF THE INVENTION

### Dual Release Embodiments

In certain embodiments, the invention provides methods and co-formulations for delivering in the co-formulation a glucose-level-controlling bioactive agent and a second bioactive agent for treating a co-morbidity of diabetes, the glucose-level-controlling bioactive agent having a first dosing regimen and the second bioactive agent having a second, distinct dosing regimen, wherein the co-formulation provides a pharmacokinetic profile of the glucose-level-controlling bioactive agent that mimics the first dosing regimen and a pharmacokinetic profile of the second bioactive agent that mimics the second dosing regimen.

The present invention provides, in one embodiment, a novel method of concurrently treating diabetes and co-morbidities, such as hypertension or dyslipidemia, by utilizing a multi-bioactive agent administration product to deliver a glucose level controlling bioactive agent, such as one which requires a controlled release dosage form, and a second bioactive agent, such as one which requires an immediate release dosage form, such as an anti-dyslipidemia bioactive agent or an anti-hypertension bioactive agent, in a single product. The multi-bioactive agent administration product of the present invention may comprise a co-formulation or a kit. An exemplary co-formulation is provided wherein a glucose-level-controlling bioactive agent is formulated as a controlled release product and an another bioactive agent is delivered in an immediate release formulation in the same overall dosage form. The multi-bioactive agent administration product of the invention may also comprise a kit, wherein each individual bioactive agent may be formulated in a separate dosage form but their presentation together in a single kit enhances patient compliance and improves the ease of bioactive agent administration over taking each bioactive agent separately from a separate bottle.

The present invention also provides, in one embodiment, a co-formulation of a first highly water soluble bioactive agent in a controlled release dosage form using a blend of hydrophobic and hydrophilic polymers to provide controlled release of the bioactive agent where needed, with a second immediate release bioactive agent, and a method of formulating such co-formulation.

While coating techniques using a core-coat combination are known in the formulation of pharmaceutical agents, these techniques have typically been applied to control the release of either a single active ingredient or as a method to prevent interaction between two bioactive agents. Thus, the invention provides an ability to deliver two such bioactive agents in a single daily dosage form or single dosage combination (in a kit), one with an otherwise typical once a day dosing regimen and another with an otherwise typical more frequent dosing during the day. This invention also provides formulating techniques to provide a single dosage form wherein the pharmacokinetic profiles of each of the included bioactive agents will mimic their respective single dose or multidose equivalents. In this way, multiple bioactive agents may be administered in a single dosage form, even when each bioactive agent has its own specific and distinct dosing regimen different from the other bioactive agent(s) in the single dosage form.

In one aspect the invention provides a co-formulation of an glucose level controlling bioactive agent with an anti-dyslipidemia bioactive agent or with another bioactive agent directed against a co-morbidity of diabetes. A glucose level controlling bioactive agent, such as for example Metformin.HCl, which needs to be dosed multiple times a day and is given in large doses, is in this embodiment formulated in controlled release form, such as, for example, controlled release beads. The co-administered second agent (e.g., anti-dyslipidemia) is, in some embodiments, required in an amount that is only a fraction of the dose of the anti-diabetic and may need to be dosed only once daily. Common problems which can be expected with co-formulating two such bioactive agents having such different dosing regimens include content uniformity, uneven layering and limiting the release of the bioactive agent requiring once a day dosing. These problems are overcome by the present invention by controlling the coating on the controlled release cores of the high dose bioactive agent, and either coating or admixing the low dose bioactive agent with the controlled release core for immediate release. The result is a single dosage form which can be administered once a day to address both conditions.

In certain embodiments, a controlled release aspect of the co-formulation utilizes, at least in part, beads comprising the bioactive agent to be released in a controlled fashion. Such beads may comprise, for example, pharmaceutically acceptable components such as water soluble gums or polymers, water insoluble polymers, polymers with pH dependent solubility, natural clays, synthetic clays, waxes, triglycerides, mixtures thereof, and the like. The selection of excipients may be sufficient to provide all or a substantial portion of the controlled release characteristics. Or, materials used to coat or embed the beads may provide all or a substantial of controlled release characteristics.

Such gums or polymers can be, for example, alginates, alkyl celluloses, hydroxyalkylcelluloses, alkyl hydroxyalkylcelluloses, carboxyalkylcelluloses, carrageenan, guar gum, agar, gum arabic, gum ghatti, gum karaya, gum tragacanth, locust bean gum, pectins, polyacrylamide, polyacrylic acid, polyethylene glycol, poly(ethylene oxide), polyvinyl alcohol, polyvinylpyrrolidone, starch, tamarind gum, xanthum gum, cross linked polyacrylic acid (Carbomers), mixtures thereof, and the like. (Alkyl in the foregoing can be, for example, C1-C3 alkyl.) Such clays can be, for example, kaolins, serpentines, smectites (montmorillonites), bentonites, illites, glauconite, chlorites, vermiculites, mixed-layer clays, attapulgite, saponite, sepiolite, synthetic clays (such as, for example, synthetic smectic clays, silicates, fluorosilicates), mixtures thereof, or the like.

Such beads may comprise, for example, pharmaceutically acceptable diluents or multifunctional excipients such as lactose, sucrose, dextrose, mannitol, sorbitol, starch, microcrystalline cellulose, dibasic calcium phosphate, calcium carbonate and calcium sulfate, including different grades of the above mentioned diluents or multifunctional excipients, coprocessed excipients such as Prosolv SMCC (silicified microcrystalline cellulose, with intimate contact and even distribution of colloidal silicon dioxide on microcrystalline cellulose surfaces, from JRS Pharma Ltd., Surrey, UK), mixtures thereof, or the like.

In embodiments where such cores are coated, coatings may be obtained from, for example, and as appropriate, polymer dispersions in aqueous solution or organic solvent. Coating methods can include, for example, fluid bed coating, pan coating, hot-melt coating in a high shear granulator, or the like. Coating polymers can include, for example, those above listed with respect to components of the core. In certain embodiments, the polymers include water insoluble polymers, or mixtures of water soluble and water insoluble polymers, or mixtures of two or more of enteric polymers, water soluble and water insoluble polymers. The coatings can include, for example, plasticizers such as, for example, acetyltributyl citrate (ATBC), acetyltriethyl citrate (ATEC), dibutyl phthalate (DBP), dibutyl sebacate (DBS), diethyl phthalate (DEP), tributyl citrate (TBC), mixtures thereof, and the like.

In certain embodiments, a more immediate release form of one or more other bioactive agents is coated over the beads (which are, for example, formulated for sustained release of one or more first bioactive agents). Or, the beads are embedded in compressed formulation of the second bioactive agent(s) and appropriate excipients for such a compression formulation.

For bead coatings intended to provide sustained release properties, a coating can provide, for example, a weight gain of from one of following lower values to one of the following upper values. Lower values can be, for example, 5, 6, 7, 8, 9, 10, and so forth by intervals of 1 to 49% w/w. Upper values can be, for example, 6, 7, 8, 9, 10, and so forth by intervals of 1 to 50% w/w.

Bead sizes after coating can be, for example, from one of following lower values to one of the following upper values. Lower values can be, for example, 75, 100, 125, 150, and so forth by intervals of 25 to 500 microns. Upper values can be, for example, 150, 175, 200, and so forth by intervals of 25 to 2000 microns. For example, ranges can be 75-2000 microns, 125-1500 microns, or 500-1200 microns. Bead size can be measured by sieve analysis.

In certain embodiments, a more sustained release form is compounded in one portion of a compression tablet or other compression product, and a more immediate release form is compounded in another portion.

Dissolution is measured in 900 ml of pH 6.8 phosphate buffer using a USP 1 dissolution apparatus at 37° C. and 100 rpm. More immediate release forms can, for example, provide 90% dissolution in 60 minutes or less, 55 minutes or less, 50 minutes or less, 45 minutes or less, 40 minutes or less, 35 minutes or less, 30 minutes or less, 25 minutes or less, or 20 minutes or less. More sustained release forms can, for example, provide a target dissolution in 2 hours or more, 2.5 hours or more, 3 hours or more, 3.5 hours or more, 4 hours or more, 5 hours or more, 6 hours or more, 7 hours or more, or 8 hours or more. The target dissolution for more sustained release can be, for example, 70% or more, 75% or more, 80% or more, 85% or more or 90% or more.

Intermediate layers in beads or in compression formulations may be used to separate compositions that are less stable or otherwise less compatible when in direct contact. Thus, a composition containing no active or a third bioactive agent may separate compositions of a first bioactive agent and of a second bioactive agent.

### Kits

Kits also provide a convenient way to deliver two or more bioactive agents targeted at treating diabetes and its co-morbidities. Kits can be of several compositions and forms which aim at providing advantages of patient compliance, improved visibility for patients with limited vision and aid in remembering the last dose taken, and patient counseling by pharmacist or physician.

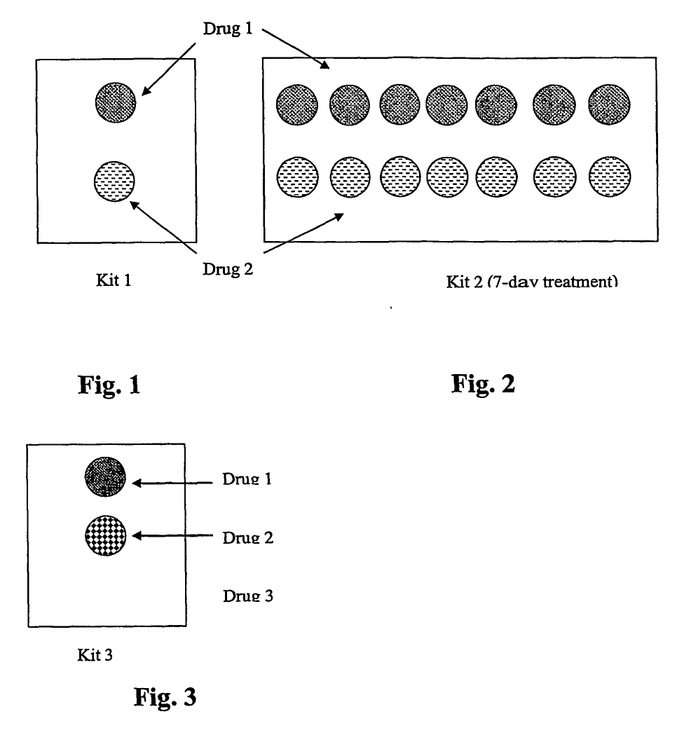

Examples of such kits include prepackaged bioactive agents as in FIGS. 1-3 or can be packaged by the pharmacists in an appropriate configuration using existing marked containers. In the kit embodiments illustrated in FIGS. 1, a first bicoactive agent 1 is contained in a first compartment 2 and a second bioactive agent 3 is contained in a second compartment 4.

The kits may be marked by the physician or pharmacist with the days of the week, as illustrated in FIG. 2, or with the days of the month to help the patient remember if she has taken her medications each day.

The kits may also be appropriately labeled to indicate whether each kit contains an individual bioactive agent to be taken more than once a day or multiple bioactive agents, each taken daily.

The kits may also be marked with “morning” and “afternoon” markings, as necessary, along with days of the week or month.

The compartments of the kits may be color coded to reflect an individual color for each single bioactive agent or multiple bioactive agent application.

The compartments of the kits may also be color coded to reflect an individual color for diabetic bioactive agent, cardiovascular bioactive agent and multi-bioactive agent combination.

The kit may be marked with detailed directions on how to take the medication to serve as a reminder to patients on proper dosing.

Each kit may contain more than two different bioactive agents, as shown in FIG. 3, which contains a first bioactive agent 1, a second bioactive agent 3, and a third bioactive agent 5.

### Definitions

The following terms shall have, for the purposes of this application, the respective meanings set forth below.

Bioactive Agent

A bioactive agent is a substance such as a chemical that can act on a cell, virus, tissue, organ or organism, including but not limited to drugs (i.e., pharmaceuticals) to create a change in the functioning of the cell, virus, organ or organism to achieve a pharmaceutical or therapeutic effect.

Co-formulation

Two or more bioactive agents are co-formulated into a co-formulation if a single dosage form contains the two or more bioactive agents. The two (or more) bioactive agents may be intimately admixed, provided no significant stability issues are thereby created. Alternatively, the two bioactive agents may be in separate initial formulations (such as particles including time release particles) so long as the initial formulations are physically linked, such as, for example, by compression or containment in a capsule. In one example, two bioactive agents can be in distinct regions of a dosage form, so long as the two regions are physically linked sufficiently so that the typical mode of administration would be concurrent. Further examples of co-formulation include suspension forms, in which the suspension form contains particles containing the two or more bioactive agents (in separate particles or the same particles), or in which one bioactive agent is in substantially particle form and another is in substantially dissolved form.

Kit

A kit of two or more bioactive agents means that the two or more bioactive agents are packaged to visually or tactilely indicate an appropriate sequence for administering the bioactive agents to a single patient.

Dosage Form

Dosage form refers to the manner in which a single unit dose of one or more bioactive agent(s) is provided for administration to a patient.

Pharmacokinetic Profile

The pharmacokinetic profile of a bioactive agent is the characteristic data for the bodily absorption, distribution, metabolism, and excretion of that bioactive agent.

Dosing Regimen

Dosing regimen refers to the proper amount of a bioactive agent and the schedule on which the bioactive agent is to be taken by a patient in need of the bioactive agent.

Dyslipidemia

Dyslipidemia refers to a disorder of lipid metabolism, and includes various conditions characterized by abnormal concentrations of one or more lipids or other associated markers (e.g., the macromolecular complexes formed by the lipid and apolipoprotein(s) that allow lipids to circulate in blood, such as Low Density Lipoproteins (LDL), Very Low Density Lipoproteins (VLDL), and Intermediate Density Lipoproteins (IDL)); apoliproteins; and the like). [Note: Cholesterol is primarily carried by Low Density Lipoproteins (LDL), and is commonly referred to as “bad” cholesterol, since elevations in LDL cholesterol correlate closely to the risk of coronary heart disease. Cholesterol is also associated with the High Density Lipoproteins (HDL), commonly referred to as “good” cholesterol, since HDL associates with cholesterol deposited in the arterial wall and atherosclerotic plaques for reverse cholesterol transport to the liver. It is therefore desirable to lower elevated levels of LDL cholesterol and to concurrently increase levels of HDL cholesterol.] Dyslipidemia includes elevated concentrations of total cholesterol, LDL cholesterol, VLDL and/or IDL cholesterol, which may be accompanied by low concentrations of HDL. Anti-dyslipidemic agents include bioactive agents that decrease the concentrations of total, LDL, VLDL and IDL cholesterol, and/or increase the concentration of HDL cholesterol.

Effective Amount

The meaning of “effective amount” will be recognized by clinicians but includes an amount effective to reduce, ameliorate or eliminate one or more symptoms of the disease sought to be treated or the condition sought to be avoided or treated, or to otherwise produce a clinically recognizable change in the pathology of the disease or condition.
treating diabetes

Treating diabetes shall include treating metabolic syndrome, obesity with propensity for diabetes, other pre-diabetic conditions and the co-morbidities of such conditions so long as the treated condition allows a meaningful interpretation for “effective amount” with respect to the bioactive agents in question.

### Exemplary Bioactive Agents

Nonlimiting examples of glucose-level-controlling bioactive agents which may be suitable for use in the present invention in therapeutically effective doses, include compounds selected from a-glucodase inhibitors (A), thiazolidinediones (B), biguanides (C), insulin, PPARα/γ agonists (D), PPARγ agonists (E), or insulin secretagogues (F) (such as, without limitation, sulfonylureas (F-1), meglitinides (F-2), and d-phenylalanine derivatives/analogs (F-3)). Further nonlimiting examples of such compounds, along with exemplary amounts for their inclusion in a formulation, include:
Acarbose (20, 50 & 100 mg) (from 10 to 200 mg) (A);
Acetohexamide (250 & 500 mg) (from 100 to 1000 mg) (F-1);
Chlorpropamide (100 & 250 mg) (from 50 to 500 mg) (F-1);
Gliclazide (80 mg) (from 40 to 160 mg) (F-1);
Glimepiride (1, 2 & 4 mg) (from 0.5 to 10 mg) (F-1);
Glipizide (2.5, 5 & 10 mg) (from 1 to 20 mg) (F-1);
Glyburide (1.25, 1.5, 2.5, 3 5 & 6 mg) (from 0.5 to 20 mg) (F-1);
Insulin (from 0.1 to 3.0 U/kg/day);
Metformin HCl (500, 850 & 1000 mg) (from 100 to 2000 mg) (C);
Miglitol (25, 50 & 100 mg) (from 10 to 200 mg) (A);
Nateglinide (60 & 120 mg) (from 30 to 200 mg) (F-3);
Pioglitazone HCl (15, 30 & 45 mg) (from 5 to 100 mg) (B);
Repaglinide (0.5, 1 & 2 mg) (from 0.2 to 5 mg) (F-2);
Rosiglitazone Maleate (2, 4 & 8 mg) (from 1 to 20 mg) (B);
Tolazamide (100, 250 & 500 mg) (from 50 to 1000 mg) (F-1);
Tolbutamide (500 mg) (from 100 to 1000 mg) (F-1);
Troglitazone (400 mg) (from 100 to 1000 mg) (B).

Peroximsome proliferators-activated receptor agonists (“dual PPAR agonists”) can be used to control hyperglycemia as well as dyslipidemia. However, it is expected that joint administration with a anti-lipemic or antihypertensive bioactive agent will be useful.

Nonlimiting examples of anti-dyslipidemia bioactive agents which may be suitable for use in the present invention (to treat a co-morbidity) in therapeutically effective doses in a formulation, include compounds selected from class HMG-CoA reductase inhibitors (G), bile acid sequestrants (H), fibric acid derivatives (I), sterols (J), cholesterol absorption inhibitors (K), MTP inhibitors (L) or nicotinic acid derivatives (M). Further nonlimiting examples of such compounds, along with exemplary amounts for inclusion in a formulation are:
Atorvastatin Calcium (10, 20, 40 & 80 mg) (from 5 to 200 mg) (G);
Cerivastatin (0.2 mg) (from 0.1 to 0.4 mg) (G);
Cholestyramine (4000 to 24000 mg as a suspension) (from 2000 to 40000 mg) (H);
Clofibrate (500 mg) (from 250 to 1000 mg) (I);
Colesevelam HCl (625 mg) (from 300 to 1200 mg) (H);
Colestipol HCl (1000 & 5000 mg) (from 500 to 10000 mg) (J);
Ezetimibe (10 mg) (from 5 to 20 mg) (K);
Febfibrozil;
Fenofibrate (54, 76, 134, 160 & 200 mg) (from 25 to 400 mg) (I?);
Fluvastatin Sodium (20, 40 & 80 mg) (from 10 to 200 mg) (G);
Gemfibrozil (600 mg) (from 300 to 1200 mg) (I?);
Lovastatin (10, 20, 40 & 60 mg) (from 5 to 100 mg) (G);
Niacin (500, 750 & 1000 mg) (from 250 to 2000 mg) (M);
Pravastatin Sodium (10, 20, 40 & 80 mg) (from 5 to 200 mg) (G);
Rosuvastatin (5, 10, 20 & 40 mg) (from 2.5 to 100 mg) (G);
Simvastatin (5, 10, 20, 40 & 80 mg) (from 2.5 to 200 mg) (G);
ZD 4522 (G);
Ciprofibrate (100 mg) (from 50 to 300 mg) (I?);
Bezafibrate (400 mg) (from 200 mg to 600 mg) (I?);
BMS-201038 (L).

Nonlimiting examples of ACE inhibitors which may be suitable for use in the present invention (to treat a co-morbidity) in therapeutically effective doses, along with exemplary amounts for their inclusion in a formulation, include:
Benazepril HCl (5, 10, 20 & 40 mg) (from 2.5 to 100 mg);
Captopril (12.5, 25, 50 & 100 mg) (from 5 to 200 mg);
Enalapril (2.5, 5, 10 & 20 mg) (from 1 to 50 mg);
Fosinopril Sodium (10, 20 & 40 mg) (from 5 to 100 mg);
Lisinopril (2.5 mg) (from 1 to 10 mg);
Moexipril HCl (7.5 & 15 mg) (from 4 to 30 mg);
Perindopril Erbumine (2, 4 & 8 mg) (from 1 to 20 mg);
Quinapril HCl (5, 10, 20 & 40 mg) (from 2.5 to 100 mg);
Ramipril (1.25, 2.5, 5 & 10 mg) (from 0.5 to 20 mg);
Trandolapril (1, 2 & 4 mg) (from 0.5 to 10 mg);

Nonlimiting examples of calcium channel blockers which may be suitable for use in the present invention (to treat a co-morbidity) in therapeutically effective doses, along with exemplary amounts for their inclusion in a formulation, include:
Amlodipine Besylate (2.5, 5 & 10 mg) (from 1 to 20 mg);
Bepridil HCl (200 & 300 mg) (from 100 to 600 mg);
Diltiazem (120 to 420 mg) (from 50 to 1000 mg);
Felodipine (2.5, 5 & 10 mg) (from 1 to 20 mg);
Isradipine (2.5, 5 & 10 mg) (from 1 to 20 mg);
Nicardipine HCl (30, 45 & 60 mg) (from 15 to 100 mg);
Nifedipine (10 to 90 mg) (from 5 to 200 mg);
Nimodipine (30 mg) (from 15 to 60 mg);
Nisoldipine (10, 20, 30 & 40) (from 5 to 100 mg);
Verapamil HCl (40 to 360 mg) (from 20 to 800 mg);

Nonlimiting examples of Beta blockers which may be suitable for use in the present invention (to treat a co-morbidity) in therapeutically effective doses, along with exemplary amounts for their inclusion in a formulation, include:
Acebutolol (5, 10, 20 & 40 mg) (from 2.5 to 100 mg);
Atenolol (50 mg) (from 20 to 150 mg);
Betaxolol HCl (10 mg) (from 5 to 20 mg);
Bisoprolol Fumarate (5 mg) (from 2.5 to 20 mg);
Carteolol HCl;
Carvedilol;
Labetolol HCl (200 mg) (from 100 to 2400 mg);
Levobunolol HCl;
Metipranolol HCl;
Metoprolol (100 mg) (from 50 to 400 mg);
Nadolol (40 mg) (from 20 to 240 mg);
Penbutolol Sulfate;
Pindolol (10 mg) (from 5 to 60 mg);
Propranolol HCl (80 mg) (from 40 to 640 mg);
Sotalol HCl;
Timolol;

Nonlimiting examples of angiotension II receptor antagonists which may be suitable for use in the present invention (to treat a co-morbidity) in therapeutically effective doses, along with exemplary amounts for their inclusion in a formulation, include:
Candesartan Cilexetil (4, 8, 16 & 32 mg) (from 2 to 100 mg);
Eprosartan Mesylate (300, 400 & 600 mg) (from 100 to 1000 mg);
Irbesartan (75, 150 & 300 mg) (from 25 to 600 mg);
Losartan Potassium (25, 50 & 100 mg) (from 10 to 200 mg);
Olmesartan Medoxomil (5, 20 & 40 mg) (from 2.5 to 100 mg);
Telmisartan (20, 40 & 80 mg) (from 10 to 200 mg);
Valsartan (80, 160 & 320 mg) (from 40 to 600 mg);

Non-limiting examples of diuretics which may be suitable for use in the present invention (to treat a co-morbidity) in therapeutically effective doses, along with exemplary amounts for their inclusion in a formulation, include:
Bendroflumethazide (5 mg) (from 1 to 10 mg);
Chlorothiazide (250 & 500 mg) (from 100 to 1000 mg);
Hydrochlorothiazide (12.5 & 50 mg) (from 5 to 100 mg);
Hydroflumethiazide (50 mg) (from 20 to 200 mg);
Methyclothiazide (2.5 & 5 mg) (from 1 to 10 mg);
Polythiazide (1, 2 & 4 mg) (from 0.5 to 10 mg);
Trichlormethiazide (4 mg) (from 1 to 10 mg);
Clorthalidone (15, 25 & 50 mg) (from 5to 100 mg);
Indapamide (1.25 & 2.5 mg) (from 0.5 to 5 mg);
Metolazone (0.5, 2.5, 5 & 10 mg) (from 0.2 to 20 mg);
Bumetanide (0.5, 1 & 2 mg) (from 0.2 to 10 mg);
Ethacrynic Acid (25 & 50 mg) (from 10 to 100 mg);
Furosemide (10, 20, 40 & 80 mg) (from 5 to 200 mg);
Torsemide (5, 10, 20 & 100 mg) (from 2.5 to 200 mg);

In certain embodiments, the group (a) bioactive agents comprise a biguanide, and a thiazolidinedione, and the co-formulation or kit optionally further comprises a statin. In certain embodiments, one of the group (a) bioactive agents is a biguanide, and one of the group (b) bioactive agents is a ACE inhibitor, and the co-formulation or kit optionally further comprises a statin. In certain embodiments, one of the group (a) bioactive agents is a biguanide, and one of the group (b) bioactive agents is a calcium channel blocker, and the co-formulation or kit optionally further comprises a statin.

In certain embodiments, one or more of the group (a) bioactive agents is a sulfonylurea, meglitinide, thiazolidinedione, biguanide or PPARγ agonist, such as, without limitation, Glimepiride, Glipizide, Repaglinide, Pioglitazone, Rosiglitazone, Troglitazone or Metformin. In certain embodiments, one or more of the group (b) bioactive agents is a HMG-CoA reductase inhibitor, fibric acid derivative or MTP inhibitor, such as, without limitation, Atorvastatin, Cerivastatin, Fluvastatin, Lovastatin, Pravastatin, Rosuvastatin, Simvastatin, Clofibrate, Fenofibrate, Febfirbozil, Ciprofibrate or Bezafibrate. In certain embodiments, one or more of the group (b) bioactive agents is a ACE inhibitor that is Captopril, Enalapril, Lisinopril or Ramipril. In certain embodiments, one or more of the group (b) bioactive agents is a calcium channel blocker that is Amlodipine, Felodipine, Nifedipine or Verapamil. In certain embodiments, one or more of the group (b) bioactive agents is a angiotension II receptor antagonist that is Irbesartan, Losartan or Valsartan.

In one embodiment of the invention, the mode of administration for which the co-formulated or co-packaged bioactive agent are formulated is oral.

### Salts

The term“pharmaceutically acceptable salts” refers to salts prepared from pharmaceutically acceptable non-toxic bases or acids including inorganic or organic bases and inorganic or organic acids. Since a given bioactive agent may be available commercially as a given salt, it is possible, and within the scope of the present invention, to provide and utilize an alternative salt-based form. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compounds used in the present invention are basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like.

It will be understood that, as used herein, references to the compounds used in the combinations described herein are meant to also include any pharmaceutically acceptable salts thereof, notwithstanding any salt designation used in its nomenclature.

### EXAMPLE 1

### Coated Cores

The following formulation L1 was used:

| Component | Percent (w/w) | Weight (mg) |
|---|---|---|
| Metformin HCl, BP | 66.7% | 534 |
| Microcrystalline Cellulose, NF | 16.7% | 133 |
| Eudragit ® NE 40D | 16.7% | 133 |
| TOTAL | 100% | 800 |

The Eudragit NE 40D is a 40% w/w aqueous dispersion of a polymer of neutral esters of acrylate and methacrylate (Rohm GmbH & Co. KG, Darmstadt, Germany). Metformin HCl and Microcrystalline Cellulose were screened and mixed. The Eudragit® NE 40D liquid was added to the above powder mix. The wet mass was mixed for additional time until uniform. The wet mass was fed into an extruder (dome type). The extrudates were further processed into beads using a spheronizer (e.g., a model MG55 from LCI Corp., Charlotte, N.C.). The beads were dried in an oven. The estimated potency of the resultant un-coated beads was 63%.

The L1 beads were coated with ethylcellulose in a bottom spray fluid bed processor. A 20% w/w dispersion of ethylcellulose was used to achieve an 18.3% (w/w) weight gain. The estimated potency of coated beads was 56%.

### EXAMPLE 2A

### Uncoated Cores

The following formulation L2 was used:

| Component | Percent (w/w) | Weight (mg) |
|---|---|---|
| Metformin HCl, BP | 69.0% | 500 |
| Microcrystalline Cellulose, NF | 17.2% | 125 |
| Eudragit ® NE 40D | 13.8% | 100 |
| TOTAL | 100% | 725 |

The cores were formed as in Example 1. The estimated potency of the resultant un-coated beads was 69%.

### EXAMPLE 2B

### Coating B

The L2 beads were coated with ethylcellulose in a bottom spray fluid bed processor. A 24% w/w dispersion of ethylcellulose was used to achieve an 8.8% (w/w) weight gain. The estimated potency of coated beads was 63%.

The 24% w/w dispersion of ethylcellulose was as follows:

| Component | Percent (w/w) |
|---|---|
| Aquacoat ® ECD-30 | 80.6% |
| DBS, NF | 5.8% |
| Purified Water, USP | 13.6% |
| TOTAL | 100% |

The Aquacoat® ECD-30 is a 30% (w/w) dispersion of ethylcellulose from FMC, Corp. The plasticizer DBS was from Morflex, Inc., Greensboro, N.C.

### EXAMPLE 2C

### Coating C

The L2 beads were coated with ethylcellulose in a bottom spray fluid bed processor. The 24% w/w dispersion of ethylcellulose (Example 2B) was used to achieve an 12.1% (w/w) weight gain. The estimated potency of coated beads was 62%.

### EXAMPLE 2D

### Coating D

The L2 beads were coated with ethylcellulose in a bottom spray fluid bed processor. The 24% w/w dispersion of ethylcellulose (Example 2B) was used to achieve an 15.9% (w/w) weight gain. The estimated potency of coated beads was 60%.

### EXAMPLE 2E

### Coating E

The L2 beads are coated with a 16% (w/w) polymer dispersion derived from Eudragit L30 D55 (a 30% (w/w) dispersion of a pH dependent anionic aqueous methacrylic/methacrylate polymer solubilizing above pH 5.5 for targeted bioactive agent delivery in the duodenum). The polymer dispersion is used to achieve, in separate batches, a 20%, 25% and 30% (w/w) weight gain.

### EXAMPLE 3

### Dissolution

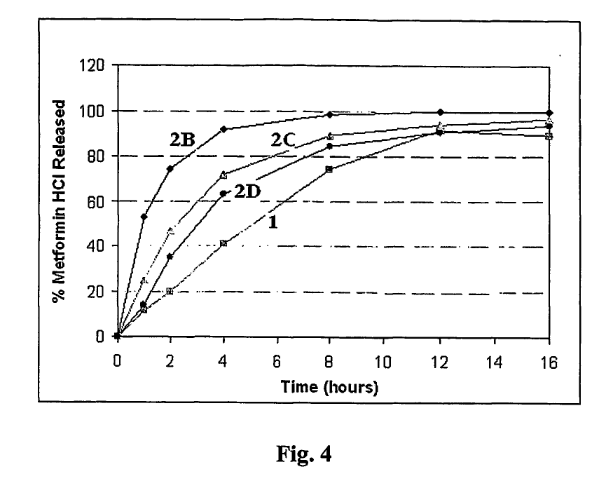

The coated cores of Examples 1, 2B, 2C and 2D were loaded into gelatin capsules and dissolution was measured in 900 ml of pH 6.8 phosphate buffer using a USP 1 dissolution apparatus at 37° C. and 100 rpm. The results are shown in FIG. 4.

### EXAMPLE 4A

### Co-formulation A

Simvastatin was suspended in a 10% Opadry® White dispersion. The simvastatin dispersion was sprayed onto cores of coated Metformin HCl beads from Example 2C in a bottom spray fluid bed processor. The beads with Simvastatin layering was finally over-coated with 10% Opadry® White dispersion (hydroxypropylmethylcellulose, polyethylene glycol, talc, titanium dioxide; from Colorcon, West Point, Pa.). The total weight gain was 6% (w/w). Capsules containing 939 mg beads (with combination bioactive agents) were filed to provide 10 mg Simvastatin and 500 mg Metfonmin HCl.

### EXAMPLE 4B

### Co-formulation B

Simvastatin was suspended in a 10% Opadry® White dispersion. The simvastatin dispersion was sprayed onto cores of Metformin HCl beads from Exarmple 1 in a bottom spray fluid bed processor. The beads with Simvastatin layering was finally over-coated with 10% Opadry® White dispersion. The total weight gain was 6% (w/w). Capsules containing 840 mg beads (with combination bioactive agents) were filed to provide 10 mg Simvastatin and 500 mg Metformin HCl.

### EXAMPLE 5

### Layered Co-Formulation

For Layer 1, the following components are used:

| Component | Percent (w/w) | Weight (mg) |
|---|---|---|
| Metformin HCl, BP | 60.0% | 500.0 |
| Hydroxyethylcellulose, USP/NF, | 5.0% | 41.7 |
| 4500 cps (Natrosol ® HHX Pharm) | | |
| Ethylcellulose, USP/NF (Ethocel ® | 14.0% | 116.7 |
| Standard 100FP Premium) | | |
| Talc, USP | 1.0% | 8.3 |
| Microcrystalline Cellulose, NF | 20.0% | 166.7 |
| (Avicel ® PH101) | | |
| Purified Water, USP | — | — |
| TOTAL | 100% | 833.3 |

All 5 ingredients are screened and mixed in a suitable low-shear blender. Water is added to the powder mix. The wet mass is mixed for additional time until a uniform wet granulation is obtained. The wet granulation is screened and dried in an oven at 40° C. overnight. The dry granulation is screened.

For Layer 2, the following components are used:

| Component | Percent (w/w) | Weight (mg) |
|---|---|---|
| Simvastatin, USP | 6.0% | 10.0 |
| Croscarmellose Sodium, NF | 2.0% | 3.3 |
| Microcrystalline Cellulose, NF | 46.0% | 76.7 |
| Dibasic Calcium Phosphate Anhydrous, | 45.0% | 75.0 |
| USP | | |
| Magnesium Stearate, USP | 1.0% | 1.7 |
| TOTAL | 100% | 166.7 |

The first four listed components are mixed until uniform. The lubricant (magnesium stearate) is mixed with the powder mix containing simvastatin.

The Layer 1 and Layer 2 powder mixes are compressed sequentially into a double layer tablet.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

1. A co-formulation or kit comprising:
(a) a pharmaceutically effective dosage of one or more a glucose-level-controlling bioactive agents selected from an α-glucodase inhibitor, sulfonylurea, meglitinide, thiazolidinediones, biguanide, insulin, dual PPARα/γ agonist, PPARγ agonist or insulin secretagogue; and
(b) a pharmaceutically effective dosage of (i) one or more of an antihypertensive bioactive agent selected from an ACE inhibitor, calcium channel blocker, beta blocker, angiotension II receptor antagonist or diuretic, or (ii) one or more of an anti-dyslipidemia bioactive agent selected from a HMG-CoA reductase inhibitor, bile acid sequestrant, fibric acid derivative, sterol, cholesterol absorption inhibitor, MTP inhibitor or nicotinic acid derivative;
wherein:
in the case of (i) a combination of a first bioactive agent of group (a) that is metformin with a second bioactive agent of group (b), or (ii) a combination of a first bioactive agent of group (a) that is a thiazolidinedione or dual PPARα/γ agonist with an angiotension II receptor antagonist, one or more of the following applies:
(I) one of the first bioactive agent or the second bioactive agent is formulated for sustained release, and the other is formulated for immediate release, each formulated for once-a-day dosing; or
(II) the co-formulation or kit comprises (A) a biguanide and a thiazolidinedione and (B) one or more group (b) bioactive agents.

2. The kit of claim 1.

3. The co-formulation of claim 1, wherein (I) applies.

4. The co-formulation of claim 3, wherein the first bioactive agent is of group (a), and the second bioactive agent is of group (b).

5. The co-formulation of claim 4, comprising a biguanide formulated for sustained release.

6. The co-formulation of claim 5, wherein the biguanide is metformin.

7. The co-formulation of claim 5, comprising a statin.

8. The co-formulation of claim 5, comprising a thiazolidinedione.

9. The co-formulation of claim 8, comprising a statin.

10. The co-formulation of claim 8, comprising an ACE inhibitor or an angiotensin II receptor antagonist.

11. The co-formulation of claim 10, comprising a statin.

12. The co-formulation of claim 8, comprising a calcium channel blocker.

13. The co-formulation of claim 12, comprising a statin.

14. The co-formulation of claim 1, comprising a capsule wherein one or more group (a) bioactive agents are formulated in sustained release beads comprised
within the capsule; and
one or more group (b) bioactive agents in a more immediate release form are comprised within the capsule.

15-23. (canceled)

24. The co-formulation of claim 14, wherein the immediate release form of group (b) bioactive agent(s) is comprised of a coating on the beads.

25. The co-formulation of claim 1, comprising a compression formulation wherein
one or more group (a) bioactive agents are formulated in sustained release form comprised within a portion of the compression formulation; and
one or more group (b) bioactive agents in a more immediate release form are comprised within another portion of the compression formulation.

26-34. (canceled)

35. The co-formulation of claim 1, comprising a suspension formulation wherein
one or more group (a) bioactive agents are formulated in sustained release form comprised within particles that are suspended or adapted to be suspended in a liquid; and
one or more group (b) bioactive agents are dissolved in the liquid.

36-44. (canceled)

45. The co-formulation of claim 1, wherein one or more of the group (a) bioactive agents is a sulfonylurea, meglitinide, thiazolidinedione, biguanide or PPARγ agonist.

46. The co-formulation of claim 45, wherein one or more of the group (a) bioactive agents is Glimepiride, Glipizide, Repaglinide, Pioglitazone, Rosiglitazone, Troglitazone or Metformin.

47. The co-formulation of claim 1, wherein one or more of the group (b) bioactive agents is a HMG-CoA reductase inhibitor, fibric acid derivative or MTP inhibitor

48. The co-formulation of claim 47, wherein one or more of the group (b) bioactive agents is Atorvastatin, Cerivastatin, Fluvastatin, Lovastatin, Pravastatin, Rosuvastatin, Simvastatin, Clofibrate, Fenofibrate, Febfirbozil, Ciprofibrate or Bezafibrate.

49. The co-formulation of claim 1, wherein one or more of the group (b) bioactive agents is a ACE inhibitor that is Captopril, Enalapril, Lisinopril or Ramipril.

50. The co-formulation of claim 1, wherein one or more of the group (b) bioactive agents is a calcium channel blocker that is Amlodipine, Felodipine, Nifedipine or Verapamil.

51. The co-formulation of claim 1, wherein one or more of the group (b) bioactive agents is a angiotension II receptor antagonist that is Irbesartan, Losartan or Valsartan.

52. A method of treating diabetes comprising administering a co-formulation of claim 1.

53. A method for delivering in the co-formulation a glucose-level-controlling bioactive agent and a second bioactive agent for treating a co-morbidity of diabetes, the glucose-level-controlling bioactive agent having a first dosing regimen and the second bioactive agent having a second, distinct dosing regimen, wherein the co-formulation provides a pharmacokinetic profile of the glucose-level-controlling bioactive agent that mimics the first dosing regimen and a pharmacokinetic profile of the second bioactive agent that mimics the second dosing regimen.
